(19) United States
(12) Patent Application Publication
(10) Pub. No.: US 2011/0306638 A1
(43) Pub. Date: Dec. 15, 2011

(54) BENZISOXAZOLE PIPERIDINYL DERIVATIVES, PHARMACEUTICAL COMPOSITIONS COMPRISING THE DERIVATIVES AND THEIR USE

(75) Inventors: Jianqi Li, Xuzhou City (CN); Guan Wang, Xuzhou City (CN); Guisen Zhang, Xuzhou City (CN); Na Lv, Xuzhou City (CN); Guangjun Jiao, Xuzhou City (CN); Shicheng Liu, Xuzhou City (CN); Shixia Zhou, Xuzhou City (CN)

(73) Assignee: NHWA PHARMA. CORPORATION, Xuzhou City (CN)

(21) Appl. No.: 13/141,811

(22) PCT Filed: Dec. 22, 2009
(86) PCT No.: PCT/CN09/75842

§ 371 Date: Aug. 17, 2011

(30) Foreign Application Priority Data
Dec. 23, 2008 (CN) 200810207606.7

(51) Int. Cl. A61K 31/454; A61P 25/20; A61P 25/04; C07D 413/14; A61P 29/00

(52) U.S. Cl. 514/322; 546/198; 514/321; 546/199

(57) ABSTRACT

The invention relates to a benzisoxazolyl piperidine derivative having the following general formula, a salt or a hydrate thereof,

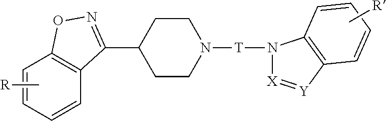

wherein R, X, Y, R′ and T are defined as in the specification. Such compounds have serotonin system modulating effects such as antagonizing effect on 5-HT_{2A} and inhibitory effect on 5-HT reuptake. The compounds have good analgesic and sedative activities with mild toxic and side effects. The invention also relates to a composition comprising said derivative and the use thereof.

### FIELD OF THE INVENTION

The present invention relates to a novel benzisoxazolyl piperidine derivative and its use in the preparation of analgesics and sedatives.

### BACKGROUND OF THE INVENTION

Tens of millions of patients are suffering from tumor pains, post-operative pains as well as various chronic and acute pains that are recurrent and severe, which is currently a big challenge for the clinic. Currently available analgesics for clinical use can be classified into three categories: 1) non-steroidal anti-inflammatory analgesics; 2) opioid analgesics; and 3) other non-opioid analgesics, mainly including local anesthetics, anti-depressants, anti-epileptics and the like. For acute pains and tumor pains, opioid analgesics or some auxiliary non-steroidal anti-inflammatory analgesics are being used clinically. However, the side effects of opioid analgesics, such as addiction, respiratory depression and decreased gastric peristalsis, have limited their application. In the treatment of various chronic non-tumor pains and neuropathic pains, the therapeutic effects of opioid analgesics or non-steroidal anti-inflammatory analgesics are rarely satisfactory. Therefore, seeking for broad-spectrum analgesics which can not only maintain potent analgesic effect but also overcome many side effects of opioid analgesics and non-steroidal anti-inflammatory analgesics, and at the same time is safe for clinical use has become a main research objective in the analgesic field.

In recent years, it has been found in clinical application that some drugs for the treatment of depression, epilepsy and anesthesia have good therapeutic effects on the relief of the pains described above. It has been confirmed that selective serotonin reuptake inhibitors (SSRIs) are effective in many pain indication tests performed on various animals as well as human beings. Currently, new analgesic indications of antidepressant Dutoxetine have been approved for marketing for the treatment of pain such as diabetic neuropathic pain, skeletal muscular pain, fibromyalgia and the like. Extensive evidence has indicated that SSRIs can not only increase the effects of traditional opioid analgesics but also have obvious effects on acute pains, inflammatory pains and neuropathic pains in various animal models [see, for example, Hynes et. al., 1975, Psychopharmacol. Commun, 1, pp 511-521; Sawynok et. al., 1999, Pharmacol. Toxicol, 85, pp 263-268; Pain, Vol. 85, pp 311-312 (2000); and Expert Opinion on Drug Discovery, Vol. 2, pp 169-184 (2007)].

In recent years, it has been reported in many literatures that endogenous 5-HT can produce various nociceptions by acting on 5-HT_{2A} and 5-HT_{2C} receptors in peripheral nervous tissues. Consequently, using 5-HT_{2A} antagonists or inverse agonists can effectively inhibit various pains especially acute inflammatory pain and hyperalgesia caused by various reasons (Neurochemistry International, Vol. 47(6), pp 394-400 (2005); Neuroscience, Vol. 130(2), pp 465-474 (2005); Pain, Vol. 122, pp 130-6 (2006); European Journal of Pain (2008), In Press, Corrected Proof, Available online 24 July, etc).

As a 5-HT reuptake inhibitor and 5-HT_{2A} antagonist, Nefazodone with dual effects was once subjected to clinical study as an analgesic. In some recent research, it has been found that coadministration of 5-HT reuptake inhibitor Paroxetine and 5-HT_{2A} antagonist Ketanserine results in increased analgesic effect of Paroxetine in animal models [(J. Pharmacol. Sci. Vol. 97, pp 61-66 (2005)].

However, currently developed 5-HT reuptake inhibitors and 5-HT_{2A} antagonists still suffer from some drawbacks in regard to their analgesic effects as well as toxic and side effects. For example, although Trazodone, a 5-HT reuptake inhibitor and 5-HT_{2A} antagonist, has established effects on some persistent pains and superior clinical effect to Ibuprofen, its therapeutic effect on severe chronic pains is poor and far from meeting the needs for clinical treatment; and Nefazodone has already been withdrawn from the market because of its severe hepatic toxicity. Therefore, there is still a need to continuously develop a novel 5-HT reuptake inhibitor and 5-HT_{2A} antagonist which can maintain potent analgesic effect and at the same time is safe for clinical use to meet the clinical needs.

### SUMMARY OF THE INVENTION

The present invention provides a novel benzisoxazolyl piperidine derivative which overcomes the disadvantages in prior art and meets clinical needs.

According to one aspect of the present invention, there is provided a benzisoxazole piperidinyl derivative having the following general formula, a salt or a hydrate thereof.

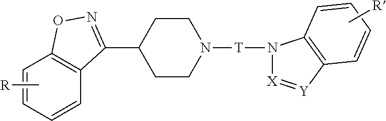

in which:

R represents H, halogen, unsubstituted or halogen substituted C₁-C₄ alkyl or unsubstituted or halogen substituted C₁-C₄ alkoxy;

X and Y represent independently CH or N;

R′ represents H, halogen, cyano, C₁-C₄ alkyl unsubstituted or substituted with halogen or cyano, C₁-C₄ alkoxy unsubstituted or substituted with halogen or cyano, or —C(═O)R₁, wherein R₁ represents H, C₁-C₄ alkyl or C₁-C₄ alkoxy; and

T represents a saturated or unsaturated, linear or branched carbon chain linking group having 1-10 carbon atoms, wherein any carbon atom of the carbon chain linking group can be replaced with one or more oxygen or sulfur atoms.

According to a further aspect of the present invention, there is provided a pharmaceutical composition comprising the benzisoxazolyl piperidine derivative according to the present invention, a salt or a hydrate thereof and a pharmaceutically acceptable carrier.

According to another aspect of the present invention, there is provided the use of a benzisoxazole piperidinyl derivative according to the present invention, a salt or a hydrate thereof in the preparation of analgesics and sedatives.

According to still another aspect of the present invention, there is provided a method for treating pains in mammals, comprising administering the benzisoxazolyl piperidine derivative according to the present invention, a salt or a hydrate thereof to an individual with such need.

### DETAILED DESCRIPTION OF THE INVENTION

In the present invention, the term “C₁-C₄ alkyl” is intended to mean linear or branched alkyl having 1 to 4 carbon atoms, such as methyl, ethyl, n-propyl, isopropyl, n-butyl, iso-butyl and tert-butyl.

The term “C₁-C₄ alkoxy” is intended to mean —O—C₁₋₄ alkyl, wherein C₁-C₄ alkyl is defined as above.

As used herein, the term “halogen” refers to fluorine, chlorine, bromine and iodine.

In the present invention, the term “mammal” includes human beings.

The benzisoxazolyl piperidine derivative according to the present invention is a compound having the following general formula:

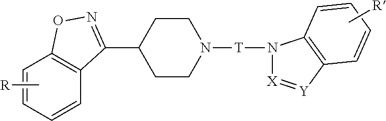

wherein, R, R′, X, Y and T are defined as above.

According to one embodiment of the benzisoxazolyl piperidine derivative of the present invention, R′ may be H, halogen or C₁-C₄ alkoxy, preferably H, F or OCH₃.

According to one embodiment of the benzisoxazolyl piperidine derivative of the present invention, R′ may be H, halogen, cyano or —C(═O)R₁, wherein R₁ represents H, C₁-C₄ alkyl or C₁-C₄ alkoxy. Preferably, R₁ represents H, Cl, F, CN or COOCH₃.

According to one embodiment of the benzisoxazolyl piperidine derivative of the present invention, T represents a saturated, linear or branched carbon chain linking group having 2-7 carbon atoms, wherein any carbon atom of the carbon chain linking group can be replaced with one or more oxygen or sulfur atoms, preferably with one or two oxygen or sulfur atoms.

The compound according to the invention can be used in the form of a free base, a pharmaceutically acceptable salt or a hydrate thereof. The salt can be an acid addition salt, for example formed from a suitable inorganic acid or organic acid. Examples of suitable inorganic acid include hydrochloric acid, sulphuric acid, hydrobromic acid, trifluoroacetic acid and phosphoric acid. Examples of suitable organic acids include carboxylic acid, phosphonic acid, sulfonic acid or aminosulfonic acid, such as acetic acid, propionic acid, octanoic acid, decoic acid, lauric acid, glycolic acid, lactic acid, 2-hydroxybutyric acid, gluconic acid, glucose monocarboxylic acid, fumaric acid, succinic acid, hexanedioic acid, heptanedioic acid, octanedioic acid, nonanedioic acid, malic acid, tartaric acid, citric acid, glucaric acid, galactaric acid; amino acids such as glutamic acid, aspartic acid, N-methylglycine, acetylaminoacetic acid, N-acetylasparagine and N-acetylcysteine; pyruvic acid, acetoacetic acid, phosphoserine, 2- or 3-glycerophosphoric acid, glucose-6-phosphate, glucose-1-phosphate, fructose-1,6-diphosphate, maleic acid, hydroxymaleic acid, methylmaleic acid, cyclohexanecarboxylic acid, adamantanecarboxylic acid, benzoic acid, salicylic acid, 1- or 3-hydroxy-naphthyl-2-carboxylic acid, 3,4,5-trimethoxy benzoic acid, 2-phenoxybenzoic acid, 2-acetoxybenzoic acid, 4-aminosalicylic acid, o-phthalic acid, phenylacetic acid, phenylglycolic acid, cinnamic acid, glucuronic acid, galacturonic acid, methanesulfonic acid or ethanesulfonic acid, 2-hyroxyethane sulfonic acid, ethane-1,2-disulfonic acid, 2-, 3- or 4-methylbenzenesulfonic acid, methylsulphuric acid, ethylsulphuric acid, dodecylsulfuric acid, methanesulfonic acid, N-cyclohexylaminosulfonic acid, N-methyl, N-ethyl or N-propyl-aminosulfonic acid, or other organic acids, such as antiscorbic acid. Among them, the salt is preferably a hydrochloride, hydrobromide, trifluoroacetate or methanesulfonate, more preferably hydrochloride or hydrobromide.

According to one embodiment of the present invention, the salt contains 0.5-3 molecules of crystal water per molecule.

According to the present invention, the benzisoxazolyl piperidine derivative is a compound selected from the group consisting of
II-1 3-(1-(2-(1H-indol-1-yl)ethyl)piperidin-4-yl)-6-fluorobenzo[d]isoxazole,
II-2 3-(1-(3-(1H-indol-1-yl)propyl)piperidin-4-yl)-6-fluorobenzo[d]isoxazole,
II-3 3-(1-(4-(1H-indol-1-yl)butyl)piperidin-4-yl)-6-fluorobenzo[d]isoxazole,
II-4 3-(1-(5-(1H-indol-1-yl)pentyl)piperidin-4-yl)-6-fluorobenzo[d]isoxazole,
II-5 6-fluoro-3-(1-(3-(6-fluoro-1H-indol-1-yl)propyl)piperidin-4-yl)benzo[d]isoxazole,
II-6 1-(3-(4-(6-fluorobenzo[d]isoxazol-3-yl)piperidin-1-yl)propyl)-1H-indole-6-carbonitrile,
II-7 methyl 1-(3-(4-(6-fluorobenzo[d]isoxazol-3-yl)piperidin-1-yl)propyl)-1H-indole-6-carboxylate,
II-8 6-fluoro-3-(1-(4-(6-fluoro-1H-indol-1-yl)butyl)piperidin-4-yl)benzo[d]isoxazole,
II-9 1-(4-(4-(6-fluorobenzo[d]isoxazol-3-yl)piperidin-1-yl)butyl)-1H-indole-6-carbonitrile,
II-10 3-(1-(4-(6-chloro-1H-indol-1-yl)butyl)piperidin-4-yl)-6-fluorobenzo[d]isoxazole,
II-11 3-(1-(3-(1H-benzo[d]imidazol-1-yl)propyl)piperidin-4-yl)-6-fluorobenzo[d]isoxazole,
II-12 3-(1-(4-(1H-benzo[d]imidazol-1-yl)butyl)piperidin-4-yl)-6-fluorobenzo[d]isoxazole,
II-13 3-(1-(3-(1H-indazol-1-yl)propyl)piperidin-4-yl)-6-fluorobenzo[d]isoxazole,
II-14 1-(4-(4-(6-fluorobenzo[d]isoxazol-3-yl)piperidin-1-yl)butyl)-1H-indazole-6-carbonitrile,
II-15 1-(4-(4-(benzo[d]isoxazol-3-yl)piperidin-1-yl)butyl)-1H-indole-6-carbonitrile,
III-1 6-fluoro-3-(1-(3-(6-fluoro-1H-benzo[d]imidazol-1-yl)propyl)piperidin-4-yl)benzo[d]isoxazole,
III-2 6-fluoro-3-(1-(4-(6-fluoro-1H-benzo[d]imidazol-1-yl)butyl)piperidin-4-yl)benzo[d]isoxazole,
III-3 1-(4-(4-(6-fluorobenzo[d]isoxazol-3-yl)piperidin-1-yl)butyl)-1H-benzo[d]imidazole-6-carbonitrile,
IV-1 1-(2-(4-(6-fluorobenzo[d]isoxazol-3-yl)piperidin-1-yl)ethoxy)-1H-indole-6-carbonitrile,
IV-2 1-(3-(4-(6-fluorobenzo[d]isoxazol-3-yl)piperidin-1-yl)propoxy)-1H-indole-6-carbonitrile,
IV-3 3-(1-(2-(6-chloro-1H-benzo[d][1,2,3]triazol-1-yloxy)ethyl)piperidin-4-yl)-6-fluorobenzo[d]isoxazole,
   or
IV-4 3-(1-(3-(6-chloro-1H-benzo[d][1,2,3]triazol-1-yloxy)propyl)piperidin-4-yl)-6-fluorobenzo[d]isoxazole,
   or a salt or a hydrate thereof.

The structural formulas of the compounds described above are shown in the following table:

| Designation | Chemical structure |
|---|---|
| II-1 | 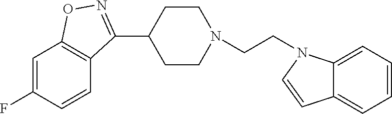 |
| II-2 | 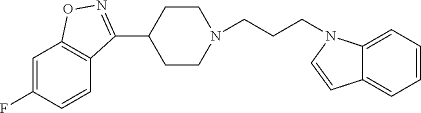 |
| II-3 | 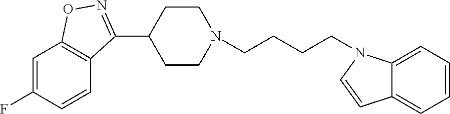 |
| II-4 | 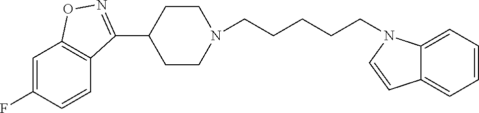 |
| II-5 | 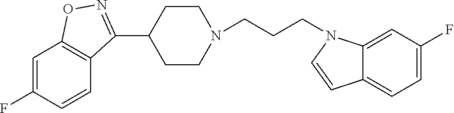 |
| II-6 | 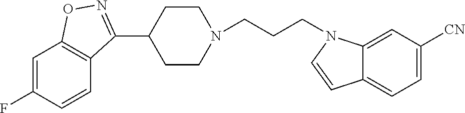 |
| II-7 | 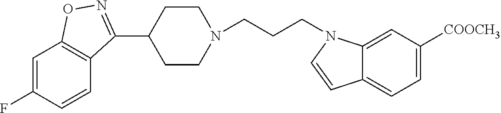 |
| II-8 | 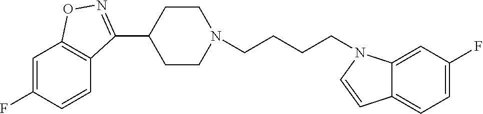 |
| II-9 | 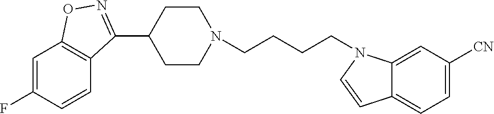 |
| II-10 | 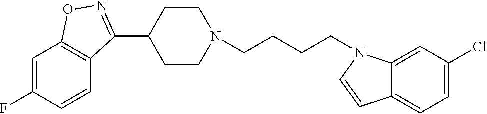 |
| II-11 | 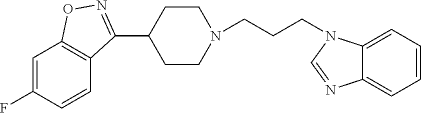 |
| II-12 | 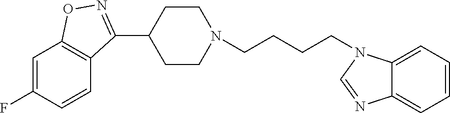 |
| II-13 | 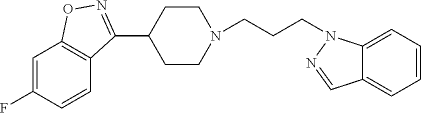 |
| II-14 | 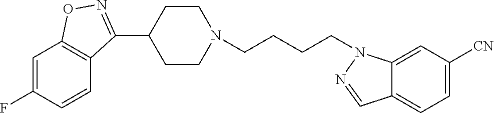 |
| II-15 | 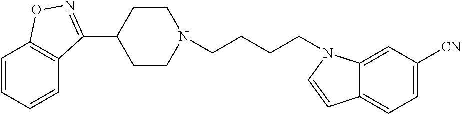 |
| III-1 | 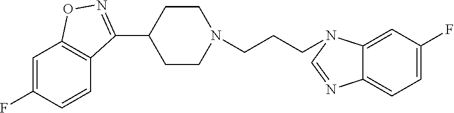 |
| III-2 | 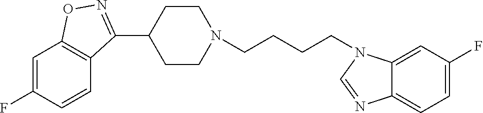 |
| III-3 | 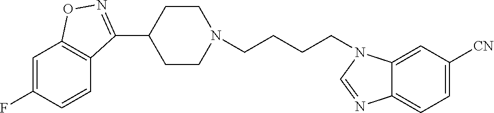 |
| IV-1 | 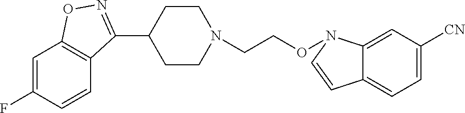 |
| IV-2 | 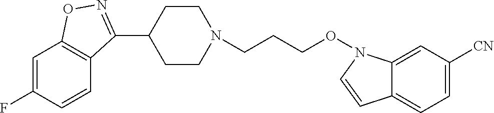 |
| IV-3 | 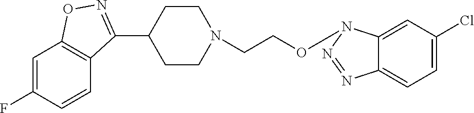 |
| IV-4 | 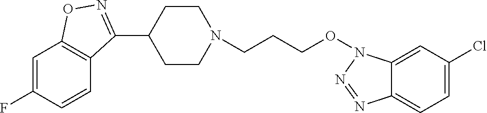 |

The compound described above is preferably a compound selected from the group consisting of
II-8 6-fluoro-3-(1-(4-(6-fluoro-1H-indol-1-yl)butyl)piperidin-4-yl)benzo[d]isoxazole,
II-9 1-(4-(4-(6-fluorobenzo[d]isoxazol-3-yl)piperidin-1-yl)butyl)-1H-indole-6-carbonitrile, or
III-2 6-fluoro-3-(1-(4-(6-fluoro-1H-benzo[d]imidazol-1-yl)butyl)piperidin-4-yl)benzo[d]isoxazole,

or a salt or a hydrate thereof.

The compound can be synthesized by using the following methods:

1. The key intermediate, substituted-3-(4-piperidinyl)benzisoxazole, in the general formula of the compound according to the invention has a general formula of:

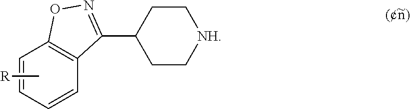

Compound (I) can be prepared by conventional synthetic methods, such as the method disclosed in U.S. Pat. No. 4,804,663, and is also commercially available.

2. The compound described herein can be prepared by the following methods:

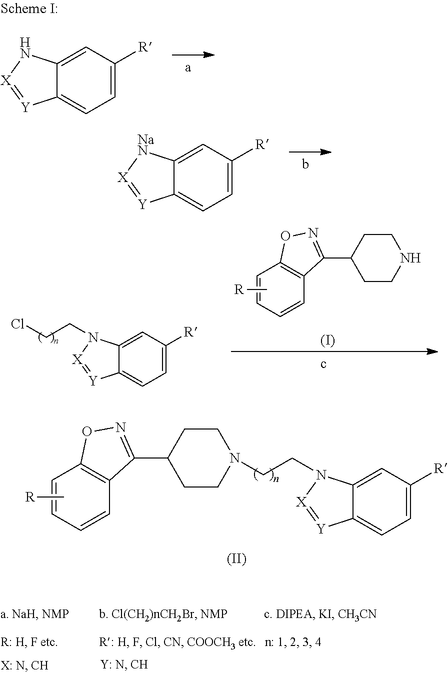

A substituted benzo-5-membered azacycle compound is used as a starting material. First, the reactive hydrogen is exchanged with sodium hydride to obtain corresponding sodium salt, which is reacted with chloroalkyl bromide to give corresponding chloride. Then the chloride is refluxed with the compound (I) in acetonitrile in the presence of diisopropylethylamine and potassium iodide (DIPEA/KI) for 6-18 hours to give a condensed product (II).

Target compounds II-1 to II-5 can be obtained using the steps described above.

### General Guidelines for Scheme I

### Abbreviated as General Procedure I

### Preparation of Benzisoxazolyl Piperidine Compound (II) Hydrochloride

Substituted indolyl (benzimidazole or benzopyrazole) (0.01 mol) is dissolved in 10 ml NMP, and sodium hydride (0.01 mol, 50% by weight) in admixture with solid paraffin is added in portions and stirred for 0.5 hour. Chloroalkyl bromide (0.015 mol) is dissolved in 5 ml NMP and the resulting mixture is added to the solution described above. A reaction is allowed to proceed at room temperature for 12 hours. The reaction solution is poured into 50 ml water and extracted with ethyl acetate (3×30 ml). The organic phases are combined, washed with 20 ml water and dried by addition of anhydrous magnesium sulfate, filtered and evaporated to remove the solvent. The residue is purified by Al₂O₃ column chromatography, eluted with petroleum ether/dichloromethane, and concentrated, obtaining corresponding chloride. The yield is 80˜85%.

The resulting chloride (0.0055 mol), the compound (I) (0.005 mol), KI (0.005 mol) and diisopropylethylamine (DIPEA) (0.02 mol) are added to 30 ml acetonitrile solution, and heated to reflux, allowing a reaction to proceed for 8-16 hours. Evaporation under reduced pressure is conducted to remove the solvent. The residue is purified by Al₂O₃ column chromatography, eluted with dichloromethane/methanol. The eluent is concentrated to dryness, and then dissolved in 30 ml ethyl acetate, is adjusted to pH of <3 with HCl/C₂H₅OH(5N). The resulting solid precipitate is filtered and recrystallized with ethyl acetate/ethanol to give hydrochloride salt of target compound (II). The yield is 60˜70%.

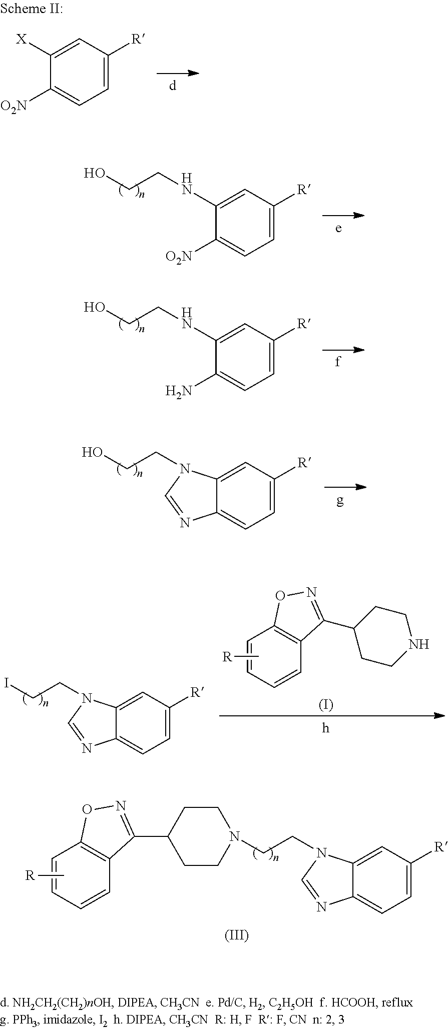

Substituted o-halonitrobenzene, used as a starting material, is first subjected to nucleophilic substitution reaction with amino alkanol. Following reduction of the nitro group into an amino group, cyclization under refluxing in formic acid is carried out to give corresponding benzimidazolylalkanol. The hydroxyl group is iodinated under the catalysis of triphenylphosphine to give corresponding iodide. The resulting iodide and the compound (I) are refluxed in acetonitrile for 6-18 hours in the presence of DIPEA/KI to give a condensed product (III).

Target compounds III-1 to III-3 can be obtained by using the steps describe above.

### General Guidelines for Scheme II

### Abbreviated as General Procedure II

### Preparation of Benzisoxazolyl Piperidine Compound (III) Hydrochloride

Substituted o-halonitrobenzene (25.2 mmol), corresponding amino alkanol (30.2 mmol) and DIPEA (60.35 mmol) are dissolved in 50 ml acetonitrile, stirred at room temperature for 72 hours, and evaporated under reduced pressure to remove the solvent. The residue is then dissolved in 50 ml dichloromethane, washed with water (20 ml×2) and saturated brine. Removal of solvent by evaporation gives corresponding o-nitrophenylaminoalkanol.

The o-nitrophenylaminoalkanol (0.02 mmol) is dissolved in 120 ml ethanol (95% by weight), and palladium on carbon (0.4 g, 5% by weight) is added under stirring. The resulting mixture is placed in a shake flask and hydrogen is introduced to allow a reaction to proceed for 1 hour until there is no more hydrogen consumption. The reaction mixture is filtered and evaporated to give an oily product, which is mixed with 15 ml 96% formic acid and heated to reflux for 2.5 hours. The reaction mixture is cooled to room temperature and 15 ml water is added. To the resulting mixture 30 ml aqueous sodium hydroxide solution (40% by weight) is slowly added under cooling by ice water, and stirred for 2 hours. The reaction mixture is extracted with dichloromethane (30 ml×3). The organic phases are combined, washed with 20 ml water and then 20 ml saturated brine. The organic phase is dried and evaporated to remove the solvent, obtaining crude oily product, which is then purified by silica column chromatography and eluted with dichloromethane/methanol to give corresponding benzimidazolylalkanol. The yield is 65˜70%.

Triphenylphosphine (4.16 mmol) and imidazole (4.16 mmol) are dissolved in 15 ml dichloromethane, then iodine (4.16 mmol) is added and stirred at room temperature to allow a reaction to proceed for 20 minutes. The resulting benzimidazolylalkanol (3.2 mmol) is dissolved in 5 ml dichloromethane and added dropwise into the reaction solution described above, allowing a reaction to proceed under stirring for 20 hours. 20 ml water is added, and stirred for 10 minutes. The aqueous layer is extracted with dichloromethane. The organic phases are combined, washed with 5% aqueous sodium bisulfate solution (2×10 ml) and then 20 ml saturated brine, dried, and purified by silica column chromatography, obtaining benzimidazolyl alkyliodide. The yield is 85˜90%.

The resulting iodide (0.0055 mol), the compound (I) (0.005 mol) and DIPEA (0.02 mol) are added to 30 ml acetonitrile solution, heated to reflux, allowing a reaction to proceed for 8-16 hours. Evaporation under reduced pressure is conducted to remove the solvent. The residue is purified by Al₂O₃ column chromatography, and eluted with dichloromethane/methanol. The eluent is concentrated to dryness and dissolved in 30 ml ethyl acetate, adjusted to pH of <3 with HCl/C₂H₅OH (5N). The resulting solid precipitate is filtered and recrystallized with ethyl acetate/ethanol to give hydrochloride salt of target compound (III). The yield is 60˜70%.

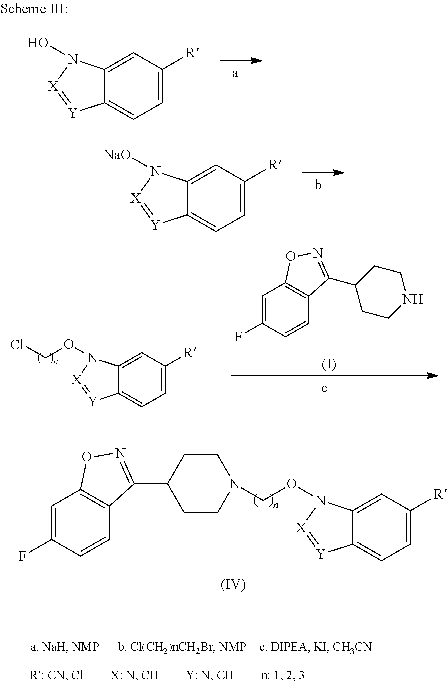

Substituted benzoazacyclophenol or substituted naphthol is used as a starting material. The reactive hydrogen of phenol hydroxyl group is exchanged with sodium hydride to give corresponding sodium salt, which is in turn reacted with chloroalkyl bromide to give corresponding chloride. The resulting chloride and the compound (I) are refluxed in acetonitrile for 6-18 hours in the presence of DIPEA/KI to give a condensed product (IV).

Target compounds IV-1 to IV-4 can be obtained by using the steps describe above.

### General Guidelines for Scheme III

### Abbreviated as General Procedure III

### Preparation of Benzisoxazolyl Piperidine Compound (IV) Hydrochloride

Substituted N-hydroxybenzo-5-membered azacycle (0.01 mol) is dissolved in 10 ml NMP, and sodium hydride (0.01 mol, 50% by weight) in admixture with solid paraffin is added in portions, and stirred to allow a reaction to proceed for 0.5 hour. Chloroalkyl bromide (0.015 mol) is dissolved in 5 ml NMP and added to the solution described above, allowing a reaction to proceed at room temperature for 12 hours. The reaction solution is poured into 50 ml water and extracted with ethyl acetate (3×30 ml). The organic phases are combined, washed with 20 ml water, dried by addition of anhydrous magnesium sulfate, filtered and evaporated to remove the solvent. The residue is purified by Al₂O₃ column chromatography, eluted with petroleum ether/dichloromethane, and concentrated, obtaining corresponding chloride. The yield is 80˜85%.

The resulting chloride (0.0055 mol), the compound (I) (0.005 mol), KI (0.005 mol) and DIPEA (0.02 mol) are added to 30 ml acetonitrile solution. The mixture is heated to reflux, allowing a reaction to proceed for 8-16 hours. Evaporation under reduced pressure is conducted to remove the solvent. The residue is purified by Al₂O₃ column chromatography, and eluted with dichloromethane/methanol. The eluent is concentrated to dryness, dissolved in 30 ml ethyl acetate, and adjusted to pH of <3 with HCl/C₂H₅OH (5N). The resulting solid precipitate is filtered and recrystallized with ethyl acetate/ethanol to give hydrochloride salt of target compound (IV). The yield is 60˜70%.

Animal trials have demonstrated that benzisoxazolyl piperidine compounds according to the invention exhibit potent effects against pain induced writhing response in mouse model of chemically induced pain, and have analgesic and sedative effects. Hot plate pharmacological tests performed on mice have also demonstrated that the compounds have analgesic effects.

Results of animal model study have indicated that II-9 has obvious analgesic effect and can be well absorbed through oral administration. No drug tolerance is observed for II-9 after repeated dosing and the potential for addiction is rather low. With negative result in Ames test and high therapeutic index, II-9 has the potential to be developed as a novel non-addictive analgesic.

The inventors have found that the derivative of the invention has reduced toxicity and mild neurological side effect.

The results described above have demonstrated that the benzisoxazolyl piperidine derivative of the invention can be used in the preparation of analgesics and sedatives.

One embodiment of the invention comprises the use of the benzisoxazolyl piperidine derivative in the preparation of anagesics.

The benzisoxazolyl piperidine derivative of the invention can also be used in the preparation of drugs for other central nervous system disorders, such as drugs for neuropathic pain, mania, anxiety, various depressions, schizophrenia, Parkinson's disease (PD), Humtington's chorea (HD), Alzheimer's Disease, senile dementia, dementia in Alzheimer's disease, memory disorder, loss of executive function, vascular dementia and other dementias, as well as dysfunction diseases related to intelligence, learning or memory.

The derivative of the invention can be administered to patients in the form of a composition through oral administration, injection and the like. The daily dosage usually is 0.1˜1 mg/kg (oral administration) or 0.02˜0.5 mg/kg (injection), which is determined by the doctor based on results of clinical tests as well as the patient's condition, age and the other factors.

The composition comprises a therapeutically effective amount of benzisoxazolyl piperidine derivative according to the invention, salt or hydrate thereof, and a pharmaceutically acceptable carrier.

The carrier can be any carrier conventionally used in pharmaceutical field, including diluent, excipient such as water; binder such as cellulose derivative, gelatin, polyvinyl pyrrolidone; bulking agent such as starch; disintegrant such as calcium carbonate, sodium bicarbonate; lubricant such as calcium stearate or magnesium stearate. Additionally, other auxiliary agents such as flavoring agent and sweetening agent can also be incorporated into the composition. When used in oral administration, it can be prepared into conventional solid formulation such as tablet, powder or capsule; when used in injection, it can be prepared into injection solution.

The various dosage forms of the composition can be prepared by using conventional methods in medical and pharmaceutical fields, wherein the content of the active ingredient is 0.1%˜99.5% (by weight).

The novel benzisoxazolyl piperidine derivative of the invention and physiologically acceptable salt thereof have very useful pharmaceutical properties and can be well tolerated. They exhibit effects on central nervous system, especially inhibitory effect on selective serotonin reuptake and antagonizing effect on selective 5-HT_{2A} receptor. Such compounds have sedative effects on various pains, including various nociceptive pains, acute pain, chronic pain, neuropathic pain, psychogenic pain and mixed pain. The pains especially include, but are not limited to, post-operative pain, neuropathic pain, central pain, somatic pain, visceral pain, chronic back pain, neck and low back pain, cancer pain, inflammatory pain, diabetic neuropathic pain, sciatic pain, tension headache, cluster headache, daily chronic headache, zoster associated neuropathic pain, facial and oral neuropathic pain as well as myofascial pain syndrome, phantom limb pain, stump pain and paraplegia pain, toothache, opioid-resistant pain, post-operative pain including heart surgery and mastectomy, angina pectoris, pelvis pain, genitourinary tract pain including cystitis, inflammation of vaginal vestibule and didymalgia, premenstrual syndrome, post-stroke pain, irritable bowel syndrome, exertion and labor pain, postpartum pain, pain caused by burning and chemical injury or solarization and bone injury pain.

In addition, the benzisoxazolyl piperidine derivative and physiologically acceptable salt thereof exhibit effects on central nervous system, especially dual effects on serotonin reuptake and selective 5-HT_{2A} receptor. It can regulate the level of serotonin in synaptic cleft to exert various physiological and pharmaceutical effects and can be used as an active pharmaceutical substance, especially as an antidepressant, anxiolytic, antipsychotic and antihypertensive, and can also be used as an intermediate for the preparation of other pharmaceutically active compounds.

The benzisoxazolyl piperidine derivative and physiologically acceptable salt thereof have very useful pharmaceutical properties and can be well tolerated, especially can be used as novel analgesics and sedatives. Such compounds are non-addictive central analgesics and have mild toxic and side effects as well as high therapeutic index.

The invention will be explained in more detail with reference to the following examples. However, it should be understood that the following examples are for illustrative purposes only and are not intended to limit the scope of the invention.

### Example 1

### Preparation of 3-(1-(2-(1H-indol-1-yl)ethyl)piperidin-4-yl)-6-fluorobenzo[d]isoxazole (II-1) hydrochloride

N-(2-chloroethyl)indole was prepared from indole in accordance with the synthesis and working-up method of General Procedure I. N-(2-chloroethyl)indole (1.0 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.31 g of white crystal having a melting point of 216˜218° C. The yield was 65.5%.

Element analysis: C₂₂H₂₂FN₃O.HCl.H₂O (theoretical %: C, 63.23; H 6.03; N 10.05; Cl, 8.48; experimental % C, 63.15; H, 6.021; N, 10.08; Cl 8.51); MS: m/z 363.2 (M⁺)

¹HNMR (DMSO-d₆): δ2.21˜2.25 (m, 2H), 2.34˜2.44 (m, 2H) 3.13˜3.22 (m, 2H,), 3.43˜3.52 (m, 3H), 3.63˜3.67 (m, 2H), 4.76˜4.81 (m, 2H), 6.50˜6.52 (d, 1H, J=3.2 Hz,), 7.05˜7.09 (t, 1H, J=7.6 Hz), 7.17˜7.22 (t, 1H, J=7.6 Hz), 7.32˜7.38 (td, 1H, J=9.2 Hz, J=2.0 Hz), 7.49˜7.51 (d, 1H, J=3.2 Hz), 7.54˜7.60 (d, 1H, J=7.6 Hz), 7.70˜7.75 (m, 2H), 8.20˜8.24 (dd, 1H, J=9.2 Hz, J=3.2 Hz), 11.46 (br, 1H, HCl).

### Example 2

### Preparation of 3-(1-(3-(1H-indol-1-yl)propyl)piperidin-4-yl)-6-fluorobenzo[d]isoxazole (II-2) hydrochloride

N-(3-chloropropyl)indole was prepared from indole in accordance with the synthesis and working-up method of General Procedure I. N-(3-chloropropyl)indole (1.07 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.28 g of a white crystal having a melting point of 209˜211° C. The yield was 61.8%.

Element analysis: C₂₃H₂₄FN₃O.HCl.2H₂O (Theoretical %: C, 66.74; H, 6.02; N, 10.15; Cl, 8.57; Experimental % C, 66.70; H, 6.01; N, 10.12; Cl, 8.55); MS: m/z 377.2 (M⁺)

¹HNMR (DMSO-d₆): δ 2.12˜2.20 (m, 2H), 2.21˜2.25 (m, 2H), 2.34˜2.45 (m, 2H), 3.14˜3.22 (m, 2H), 3.42˜3.50 (m, 3H) 3.64˜3.67 (m, 2H), 4.77˜4.80 (m, 2H₂), 6.52˜8.22 (m, 9H, Ar—H), 11.20 (br, 1H, HCl).

### Example 3

### Preparation of 3-(1-(4-(1H-indol-1-yl)butyl)piperidin-4-yl)-6-fluorobenzo[d]isoxazole (II-3) hydrochloride

N-(4-chlorobutyl)indole was prepared from indole in accordance with the synthesis and working-up method of General Procedure I. N-(4-chlorobutyl)indole (1.14 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.33 g of a white crystal having a melting point of 201˜203° C. The yield was 62.1%.

MS: m/z 391.2 (M⁺)

¹HNMR (DMSO-d₆): 1.68˜1.74 (m, 2H), 1.79˜1.85 (m, 2H), 2.16˜2.21 (m, 2H), 2.27˜2.37 (m, 2H), 3.00˜3.13 (m, 4H), 3.41˜3.48 (m, 1H), 3.53˜3.57 (m, 2H), 4.20˜4.25 (t, 2H, J=6.8 Hz), 6.43 (d, 1H, J=6.4 Hz), 7.01 (t, 1H, J=7.6 Hz), 7.13 (t, 1H, J=7.6 Hz), 7.33 (td, 1H, J=9.2 Hz, J=2.0 Hz), 7.42 (d, 1H, J=3.2 Hz), 7.52 (d, 1H, J=7.6 Hz), 7.54 (d, 1H, J=7.6 Hz), 7.71 (dd, 1H, J=8.8 Hz, J=2.0 Hz), 8.20 (dd, 1H, J=8.8 Hz, J=3.2 Hz), 10.60 (br, 1H, HCl).

### Example 4

### Preparation of 3-(1-(5-(1H-indol-1-yl)pentyl)piperidin-4-yl)-6-fluorobenzo[d]isoxazole (II-4) hydrochloride

N-(5-chloropentyl)indole was prepared from indole in accordance with the synthesis and working-up method of General Procedure I. N-(5-chloropentyl)indole (1.22 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 15 hours. Working-up according to General Procedure I gave 1.41 g of a white crystal having a melting point of 181˜183° C. The yield was 63.8%.

MS: m/z 405.2 (M⁺)

¹HNMR (DMSO-d₆): δ1.45˜1.54 (m, 2H), 1.81˜1.93 (m, 4H), 2.20˜2.25 (m, 2H), 2.34˜2.46 (m, 2H), 3.15˜3.22 (m, 2H), 3.44˜3.50 (m, 3H), 3.65˜3.67 (m, 2H), 4.77˜4.82 (m, 2H), 6.50˜8.21 (m, 9H, Ar—H), 11.14 (br, 1H, HCl).

### Example 5

### Preparation of 6-fluoro-3-(1-(3-(6-fluoro-1H-indol-1-yl)propyl)piperidin-4-yl)benzo[d]isoxazole (II-5) hydrochloride

N-(3-chloropropyl)-6-fluoroindole was prepared from 6-fluoroindole in accordance with the synthesis and working-up method of General Procedure I. N-(3-chloropropyl)-6-fluoroindole (1.16 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.37 g of a white crystal having a melting point of 211˜213° C. The yield was 63.4%.

MS: m/z 395.2 (M⁺)

¹HNMR (DMSO-d₆): δ2.10˜2.18 (m, 2H), 2.21˜2.25 (m, 2H), 2.34˜2.45 (m, 2H,), 3.14˜3.22 (m, 2H), 3.42˜3.50 (m, 3H), 3.64˜3.67 (m, 2H), 4.77˜4.80 (m, 2H), 6.53˜8.20 (m, 8H, Ar—H), 10.90 (br, 1H, HCl).

### Example 6

### Preparation of 1-(3-(4-(6-fluorobenzo[d]isoxazol-3-yl)piperidin-1-yl)propyl)-1H-indole-6-carbonitrile (II-6) hydrochloride

N-(3-chloropropyl)-6-cyanoindole was prepared from 6-cyanoindole in accordance with the synthesis and working-up method of General Procedure I. N-(3-chloropropyl)-6-cyanoindole (1.20 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.32 g of a white crystal having a melting point of 203˜205° C. The yield was 60.3%.

MS: m/z 402.2 (M⁺)

¹HNMR (DMSO-d₆): δ2.15˜2.20 (m, 2H), 2.27˜2.38 (m, 4H), 3.04˜3.11 (m, 4H), 3.40-3.44 (m, 1H), 3.58˜3.62 (m, 2H), 4.39˜4.43 (m, 2H), 6.63 (d, 1H, J=6.8 Hz), 7.31 (dd, 2H, J=9.2 Hz, J=2.0 Hz), 7.37 (d, 1H, J=8.4 Hz), 7.70˜7.75 (m, 2H), 7.76 (d, 1H, J=6.8 Hz), 8.20 (dd, 1H, J=8.8 Hz, J=3.6 Hz), 8.23 (s, 1H), 10.91 (br, 1H, HCl).

### Example 7

### Preparation of methyl 1-(3-(4-(6-fluorobenzo[d]isoxazol-3-yl)piperidin-1-yl)propyl)-1H-indole-6-carboxylate (II-7) hydrochloride

N-(3-chloropropyl)-6-methoxycarbonylindole was prepared from 6-methoxy carbonylindole in accordance with the synthesis and working-up method of General Procedure I. N-(3-chloropropyl)-6-methoxycarbonylindole (1.38 g, 0.0055 mol), 4-(3-(6-fluorobenzisoxazolyl))piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.44 g of a white crystal having a melting point of 208˜210° C. The yield was 61.0%.

MS: m/z 435.2 (M⁺)

¹HNMR (DMSO-d₆): δ 2.16˜2.21 (m, 2H), 2.26˜2.38 (m, 4H), 3.05˜3.15 (m, 4H), 3.40-3.43 (m, 1H), 3.59˜3.64 (m, 2H), 3.89 (s, 3H), 4.40˜4.43 (m, 2H), 6.65˜8.22 (m, 8H), 11.02 (br, 1H, HCl).

### Example 8

### Preparation of 6-fluoro-3-(1-(4-(6-fluoro-1H-indol-1-yl)butyl)piperidin-4-yl) benzo[d]isoxazole (II-8) hydrochloride

N-(4-chlorobutyl)-6-fluoroindole was prepared from 6-fluoroindole in accordance with the synthesis and working-up method of General Procedure I. N-(4-chlorobutyl)-6-fluoroindole (1.24 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.38 g of a white crystal having a melting point of 198˜200° C. The yield was 61.9%.

MS: m/z 409.2 (M⁺)

¹HNMR (DMSO-d₆): 1.69˜1.75 (m, 4H), 2.18˜2.36 (m, 4H), 3.01˜3.15 (m, 4H), 3.41˜3.47 (m, 1H), 3.55˜3.58 (m, 2H), 4.33 (t, 2H), 6.60˜8.22 (m, 8H, Ar—H), 10.76 (br, 1H, HCl).

### Example 9

### Preparation of 1-(4-(4-(6-fluorobenzo[d]isoxazol-3-yl)piperidin-1-yl)butyl)-1H-indole-6-carbonitrile (II-9) hydrochloride

N-(4-chlorobutyl)-6-cyanoindole was prepared from 6-cyanoindole in accordance with the synthesis and working-up method of General Procedure I. N-(4-chlorobutyl)-6-cyanoindole (1.28 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.45 g of a white crystal (1.45 g) having a melting point of 216˜218° C. The yield was 64.2%.

MS: m/z 416.2 (M⁺)

¹HNMR (DMSO-d₆): 1.68˜1.76 (m, 4H), 2.17˜2.36 (m, 4H), 3.01˜3.14 (m, 4H), 3.42˜3.49 (m, 1H), 3.54˜3.58 (m, 2H), 4.32 (t, 2H, J=8.4 Hz), 6.61 (d, 1H, J=2.8 Hz), 7.31-7.36 (m, 2H), 7.72 (d, 1H, J=8.4 Hz), 7.75 (d, 2H, J=2.8 Hz), 8.15˜8.22 (m, 2H), 10.53 (br, 1H, HCl).

### Example 10

### Preparation of 3-(1-(4-(6-chloro-1H-indol-1-yl)butyl)piperidin-4-yl)-6-fluorobenzo[d]isoxazole (II-10) hydrochloride

N-(4-chlorobutyl)-6-chloroindole was prepared from 6-chloroindole in accordance with the synthesis and working-up method of General Procedure I. N-(4-chlorobutyl)-6-chloroindole (1.33 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.54 g of a white crystal having a melting point of 203˜204° C. The yield was 66.7%.

MS: m/z 425.2 (M⁺)

¹HNMR (DMSO-d₆): 1.68˜1.76 (m, 4H), 2.17˜2.36 (m, 4H), 3.01˜3.14 (m, 4H), 3.42˜3.49 (m, 1H), 3.54˜3.58 (m, 2H), 4.32 (t, 2H, J=8.4 Hz), 6.61 (d, 1H, J=2.8 Hz), 7.29-7.40 (m, 2H,), 7.72 (d, 1H, J=8.4 Hz), 7.76 (d, 2H, J=2.8 Hz), 8.16˜8.24 (m, 2H), 10.80 (br, 1H, HCl).

### Example 11

### Preparation of 3-(1-(3-(1H-benzo[d]imidazol-1-yl)propyl)piperidin-4-yl)-6-fluorobenzo[d]isoxazole (II-11) hydrochloride

N-(3-chloropropyl)benzimidazole was prepared from benzimidazole in accordance with the synthesis and working-up method of General Procedure I. N-(3-chloropropyl)benzimidazole (1.07 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.32 g of a white crystal having a melting point of 252˜254° C. The yield was 63.8%.

MS: m/z 378.2 (M⁺)

¹HNMR (DMSO-d₆): δ2.15˜2.19 (m, 2H), 2.44˜2.49 (m, 2H), 3.07˜3.22 (m, 4H), 3.43˜3.49 (m, 1H), 3.61-3.65 (m, 2H), 4.70 (t, 2H, J=6.8 Hz), 7.32 (tt, 1H, J=9.2 Hz, J=2.0 Hz), 7.62 (t, 2H, J=6.8 Hz), 7.72 (dd, 1H, J=9.2 Hz, J=2.0 Hz), 7.89 (d, 1H, J=6.8 Hz), 8.13 (d, 1H, J=6.8 Hz), 8.26 (dd, 1H, J=9.2 Hz, J=3.2 Hz), 9.74 (s, 1H), 11.51 (br, 1H, HCl).

### Example 12

### Preparation of 3-(1-(4-(1H-benzo[d]imidazol-1-yl)butyl)piperidin-4-yl)-6-fluorobenzo[d]isoxazole (II-12) hydrochloride

N-(4-chlorobutyl)benzimidazole was prepared from benzimidazole in accordance with the synthesis and working-up method of General Procedure I. N-(4-chlorobutyl)benzimidazole (1.15 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.37 g of a white crystal (1.37 g) having a melting point of 205˜207° C. The yield was 63.9%.

MS: m/z 392.2 (M⁺)

¹HNMR (DMSO-d₆): δ1.83-1.86 (m, 2H), 1.97-2.08 (m, 2H), 2.15-2.19 (m, 2H,), 2.39˜2.48 (m, 2H), 3.04˜3.16 (m, 4H), 3.46˜3.50 (m, 1H), 3.57-3.61 (m, 2H), 4.56 (t, 2H, J=6.8 Hz), 7.32 (td, 1H, J=9.2 Hz, J=2.0 Hz), 7.62 (m, 2H), 7.72 (dd, 1H, J=9.2 Hz, J=2.0 Hz), 7.88 (dd, 1H, J=6.8 Hz, J=2.0 Hz), 8.07 (d, 1H, J=6.8 Hz), 8.26 (dd, 1H, J=9.2 Hz, J=3.6 Hz), 9.71 (s, 1H), 11.20 (br, 1H, HCl).

### Example 13

### Preparation of 3-(1-(3-(1H-indazol-1-yl)propyl)piperidin-4-yl)-6-fluorobenzo[d]isoxazole (II-13) hydrochloride

1-(3-chloropropyl)benzopyrazole was prepared from benzopyrazole in accordance with the synthesis and working-up method of General Procedure I. 1-(3-chloropropyl)benzopyrazole (1.07 g, 0.0055 mol), 4-(3-(6-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.30 g of a white crystal having a melting point of 201˜203° C. The yield was 62.7%.

MS: m/z 378.2 (M⁺)

¹HNMR (DMSO-d₆): δ2.10˜2.21 (m, 2H), 2.41˜2.52 (m, 2H), 3.05˜3.29 (m, 4H), 3.41˜3.65 (m, 3H), 4.68 (t, 2H, J=6.8 Hz), 7.20˜9.86 (m, 8H, Ar—H), 11.32 (br, 1H, HCl).

### Example 14

### Preparation of 1-(4-(4-(6-fluorobenzo[d]isoxazol-3-yl)piperidin-1-yl)butyl)-1H-indazole-6-carbonitrile (II-14) hydrochloride

1-(4-chlorobutyl)-6-cyanobenzopyrazole was prepared from 6-cyano benzopyrazole in accordance with the synthesis and working-up method of General Procedure I. 1-(4-chlorobutyl)-6-cyanobenzopyrazole (1.29 g, 0.0055 mol), 4-(3-(6-fluorobenzisoxazolyl))piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.43 g of a white crystal having a melting point of 189˜191° C. The yield was 63.0%.

MS: m/z 417.2 (M⁺)

¹HNMR (DMSO-d₆): δ2.09˜2.22 (m, 2H), 2.40˜2.53 (m, 2H), 3.04˜3.29 (m, 4H), 3.43˜3.67 (m, 3H), 4.71 (t, 2H, J=6.8 Hz), 7.15˜8.29 (m, 7H, Ar—H), 11.07 (br, 1H, HCl).

### Example 15

### Preparation of 1-(4-(4-(benzo[d]isoxazol-3-yl)piperidin-1-yl)butyl)-1H-indole-6-carbonitrile (II-15) hydrochloride

N-(4-chlorobutyl)-6-cyanoindole was prepared from 6-cyanoindole in accordance with the synthesis and working-up method of General Procedure I. N-(4-chlorobutyl)-6-cyanoindole (1.28 g, 0.0055 mol), 4-(3-benzisoxazolyl)piperidine (1.01 g, 0.005 mol), DIPEA (2.58 g, 0.02 mol) and KI (0.83 g, 0.005 mol) were reacted under refluxing in 30 ml acetonitrile for 12 hours. Working-up according to General Procedure I gave 1.41 g of a white crystal having a melting point of 215˜217° C. The yield was 64.8%.

MS: m/z 398.2 (M⁺)

¹HNMR (DMSO-d₆): 1.66˜1.75 (m, 4H), 2.18˜2.40 (m, 4H), 3.00˜3.14 (m, 4H), 3.42˜3.51 (m, 1H), 3.54˜3.59 (m, 2H), 4.31 (t, 2H, J=8.4 Hz), 6.82˜7.79 (m, 9H), 10.92 (br, 1H, HCl).

### Example 16

### Preparation of 6-fluoro-3-(1-(3-(6-fluoro-1H-benzo[d]imidazol-1-yl)propyl)piperidin-4-yl)benzo[d]isoxazole (III-1) hydrochloride

6-fluoro-1-(3-iodopropyl)benzimidazole was prepared from 2,4-difluoronitrobenzene in accordance with the synthesis and working-up method of General Procedure II. 6-fluoro-1-(3-iodopropyl)benzimidazole (1.67 g, 0.0055 mol), 4-(3-(6-fluorobenzisoxazolyl))piperidine (1.10 g, 0.005 mol) and DIPEA (2.58 g, 0.02 mol) were reacted under refluxing in 30 ml acetonitrile for 15 hours. Working-up according to General Procedure II gave 1.51 g of a white crystal having a melting point of 206˜208° C. The yield was 69.6%.

MS: m/z 397.2 (M⁺)

¹HNMR (DMSO-d₆): δ2.04˜2.27 (m, 2H), 2.40˜2.54 (m, 2H), 3.03˜3.30 (m, 4H), 3.41˜3.66 (m, 3H), 4.63 (t, 2H, J=6.8 Hz), 7.21˜9.82 (m, 8H, Ar—H), 10.96 (br, 1H, HCl).

### Example 17

### Preparation of 6-fluoro-3-(1-(4-(6-fluoro-1H-benzo[d]imidazol-1-yl)butyl)piperidin-4-yl)benzo[d]isoxazole (III-2) hydrochloride

6-fluoro-1-(4-iodobutyl)benzimidazole was prepared from 2,4-difluoronitrobenzene in accordance with the synthesis and working-up method of General Procedure II. 6-fluoro-1-(4-iodobutyl)benzimidazole (1.75 g, 0.0055 mol), 4-(3-(6-fluorobenzisoxazolyl))piperidine (1.10 g, 0.005 mol) and DIPEA (2.58 g, 0.02 mol) were reacted under refluxing in 30 ml acetonitrile for 15 hours. Working-up according to General Procedure II gave 1.49 g of a white crystal having a melting point of 199˜201° C. The yield was 66.5%.

MS: m/z 411.2 (M⁺)

¹HNMR (DMSO-d₆): δ1.81˜2.09 (m, 4H), 2.10˜2.48 (m, 4H), 3.04˜3.22 (m, 4H), 3.43˜3.68 (m, 3H), 4.59 (t, 2H, J=6.8 Hz), 7.27˜9.76 (m, 7H), 11.14 (br, 1H, HCl).

### Example 18

### Preparation of 1-(4-(4-(6-fluorobenzo[d]isoxazol-3-yl)piperidin-1-yl)butyl)-1H-benzo[d]imidazole-6-carbonitrile (III-3) hydrochloride

1-(3-iodobutyl)-6-cyanobenzimidazole was prepared from 2-chloro-4-cyanonitrobenzene in accordance with the synthesis and working-up method of General procedure II. 1-(3-iodobutyl)-6-cyanobenzimidazole (1.79 g, 0.0055 mol), 4-(3-(6-fluorobenzisoxazolyl))piperidine (1.10 g, 0.005 mol) and DIPEA (2.58 g, 0.02 mol) were reacted under refluxing in 30 ml acetonitrile for 15 hours. Working-up according to General Procedure II gave 1.55 g of a white crystal having a melting point of 188˜190° C. The yield was 68.1%.

MS: m/z 418.2 (M⁺)

¹HNMR (DMSO-d₆): δ1.83˜2.08 (m, 4H), 2.12˜2.53 (m, 4H), 3.03˜3.21 (m, 4H), 3.45˜3.69 (m, 3H), 4.61 (t, 2H, J=6.8 Hz), 7.25˜9.79 (m, 7H), 11.08 (br, 1H, HCl).

### Example 19

### Preparation of 1-(2-(4-(6-fluorobenzo[d]isoxazol-3-yl)piperidin-1-yl)ethoxy)-1H-indole-6-carbonitrile (IV-1) hydrochloride

N-(2-chloroethoxy)-6-cyanoindole was prepared from N-hydroxy-6-cyanoindole in accordance with the synthesis and working-up method of General Procedure III. N-(2-chloroethoxy)-6-cyanoindole (1.21 g, 0.0055 mol), 44346-fluorobenzisoxazolyl)) piperidine (1.10 g, 0.005 mol), DIPEA (2.58 g, 0.02 mol) and KI (0.83 g, 0.005 mol) were reacted under refluxing in 30 ml acetonitrile for 15 hours. Working-up according to General Procedure III gave 1.39 g of a white crystal having a melting point of 212˜214° C. The yield was 63.0%.

MS: m/z 429.2 (M⁺)

¹HNMR (DMSO-d₆): δ 2.30˜2.47 (m, 4H), 3.32˜3.53 (m, 3H), 3.76˜3.86 (m, 4H, A-H), 5.05˜5.08 (m, 2H), 7.41˜8.30 (m, 8H, Ar—H), 10.98 (br, 1H, HCl).

### Example 20

### Preparation of 1-(3-(4-(6-fluorobenzo[d]isoxazol-3-yl)piperidin-1-yl)propoxy)-1H-indole-6-carbonitrile (IV-2) hydrochloride

N-(3-chloropropoxy)-6-cyanoindole was prepared from N-hydroxy-6-cyano indole in accordance with the synthesis and working-up method of General Procedure III. N-(3-chloropropoxy)-6-cyanoindole (1.29 g, 0.0055 mol), 4-(3-(6-fluoro benzisoxazolyl))piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure III gave 1.44 g of a white crystal having a melting point of 207˜209° C. The yield was 63.3%.

MS: m/z 418.2 (M⁺)

¹HNMR (DMSO-d₆): 2.21˜2.32 (m, 2H), 2.34˜2.56 (m, 4H), 3.14˜3.53 (m, 5H), 3.64˜3.77 (m, 2H), 4.73 (t, 2H, J=6.0 Hz), 7.34˜8.22 (m, 8H, Ar—H), 11.04 (br, 1H, HCl)

### Example 21

### Preparation of 3-(1-(2-(6-chloro-1H-benzo[d][1,2,3]triazol-1-yloxy)ethyl)piperidin 4-yl)-6-fluorobenzo[d]isoxazole (IV-3) hydrochloride

N-(2-chloroethoxy)-6-chlorobenzotriazole was prepared from N-hydroxy-6-chlorobenzotriazole in accordance with the synthesis and working-up method of General Procedure III. N-(2-chloroethoxy)-6-chlorobenzotriazole (1.28 g, 0.0055 mol), 4-(3-(6-fluorobenzisoxazolyl))piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure III gave 1.41 g of a white crystal having a melting point of 208˜210° C. The yield was 62.4%.

MS: m/z 415.1 (M⁺)

¹HNMR (DMSO-d₆): δ2.33˜2.41 (m, 4H), 3.32˜3.40 (m, 2H), 3.48˜3.53 (m, 1H), 3.76˜3.78 (m, 2H), 3.82-3.86 (m, 2H), 5.06˜5.08 (m, 2H), 7.36 (tt, 1H, J=9.2 Hz, J=2.0 Hz), 7.53 (dd, 1H, J=8.8 Hz, J=2.2 Hz) 7.72˜7.76 (dd, 1H, J=9.2 Hz, J=2.0 Hz), 8.14 (d, 1H, J=8.8 Hz, J=2.2 Hz), 8.20 (dd, 1H, J=9.2 Hz, J=3.2 Hz), 8.30 (s, 1H), 11.03 (br, 1H, HCl).

### Example 22

### Preparation of 3-(1-(3-(6-chloro-1H-benzo[d][1,2,3]triazol-1-yloxy)propyl)piperidin-4-yl)-6-fluorobenzo[d]isoxazole (IV-4) hydrochloride

N-(3-chloropropoxy)-6-chlorobenzotriazole was prepared from N-hydroxy-6-chlorobenzotriazole in accordance with the synthesis and working-up method of General Procedure III. N-(3-chloropropoxy)-6-chlorobenzotriazole (1.35 g, 0.0055 mol), 4-(3-(6-fluorobenzisoxazolyl))piperidine (1.10 g, 0.005 mol), DIPEA (2.58 g, 0.02 mol) and KI (0.83 g, 0.005 mol) were reacted under refluxing in 30 ml acetonitrile for 12 hours. Working-up according to General Procedure III gave 1.57 g of a white crystal having a melting point of 218˜220° C. The yield was 67.4%.

MS: m/z 429.1 (M⁺)

¹HNMR (DMSO-d₆): 2.22˜2.30 (m, 2H), 2.34˜2.54 (m, 4H), 3.14˜3.23 (m, 2H), 3.42˜3.53 (m, 3H), 3.68˜3.72 (m, 2H), 4.71 (t, 2H, J=6.0 Hz), 7.34 (t, 1H, J=8.8 Hz), 7.51 (d, 1H, J=8.8 Hz), 7.73 (d, 1H, J=8.8 Hz), 8.12 (d, 1H, J=8.8 Hz), 8.17 (s, 1H), 8.22 (dd, 1H, J=8.8 Hz, J=3.2 Hz), 11.02 (br, 1H, HCl).

### Example 23

| | | |
|---|---|---|
| Tablet: | compound prepared in Example 1 | 25 mg |
| | sucrose | 155 mg |
| | corn starch | 65 mg |
| | magnesium stearate | 5 mg |

### Preparation Procedure:

The active ingredient, sucrose and corn starch were mixed, wetted by addition of water and well stirred. Then the resulting mixture was dried, milled and passed through a mesh. Magnesium stearate was added, mixed well and compressed into tablets. Each tablet weighed 250 mg and contained 25 mg active ingredient.

### Example 24

| | | |
|---|---|---|
| Injection: | compound prepared in Example 22 | 10 mg |
| | water for injection | 990 mg |

### Preparation Procedure:

The active ingredient was dissolved in water for injection, mixed well and filtered. The resulting solution was aliquoted into ampoules under sterile conditions. Each ampoule contained 100 mg solution and the content of the active ingredient was 1 mg/ampoule.

### Example 25

### In-Vitro Binding Between the Compounds and 5-HT_{2A} Receptor

### 1. Test Sample

All test samples were dissolved in DMSO at a concentration of 0.01 mol/L, and then diluted with deionized water to 100 umol/L.

### 2. Experimental Materials

### 1) Cellular Transformation of 5-HT_{2A}

HEK293 cells were transformed with plasmid carriers containing the gene coding for 5-HT_{2A} receptor protein by using calcium phosphate transformation. Stable cell strains which could stably express 5-HT_{2A} receptor protein were obtained from the transformed cells by cultivating in a culture media containing G418, selecting monoclones of the cell and performing radioactive ligand binding assay.

### 2) Experimental Materials for Receptor Binding

Isotope ligand [³H]-Ketanserin (67.0 Ci/mmol) was purchased from PerkinElmer Ltd; (+)spiperone was purchased from RBI company; GF/C glass fiber filters were purchased from Whatman company; Tris was imported to load separately; PPO, POPOP were purchased from Shanghai first reagent factory; lipid-soluble scintillation fluid. Beckman LS 6500 Scintillation Counter.

### 3. Assay

### 1) Receptor Competitive Binding Assay

HEK-293 cells were infected with recombined viruses containing various genes described above. 48-72 hours later, receptor proteins were abundantly expressed on the cell membrane. Cells were centrifuged at 1000 rpm for 5 min Culture media was discarded, and cell bodies were collected and stored at −20° C. in a freezer. The cell bodies were resuspended with Tris-HCl reaction buffer during assay.

Receptor competitive binding assay: 10 μl test compound, 10 μl radioactive ligand and 80 μl receptor protein were added into a test tube, so that the final concentrations of both test compound and positive drug were 10 μmol/L. After incubation at 37° C. for 15 min, the reaction mixture was immediately transferred into ice bath to terminate the reaction; the reaction mixture was quickly filtered through GF/C glass cellulose filter on a Millipore cell sample collector, and was then washed with eluting solution (50 mM Tris-HCl, PH 7.7, 3 ml×3) and dried in a microwave for 8-9 min. The filter was transferred into a 0.5 ml centrifuge tube, and 500 μl lipid soluble scintillation fluid was added. The centrifuge tube was allowed to stand in darkness for more than 30 min. Radioactivity was determined by counting Inhibition percentage of each compound on isotope ligand binding was calculated according to the following equation:

inhibition percentage(I %)=cpm of overall binding tube−cpm of compound/cpm of overall binding tube−cpm of nonspecific binding tube×100%.

Each compound was tested in two duplicate test tubes and two separate tests were performed.

### 4. Results

1) Results from Preliminary Screening

TABLE 1
Inhibition of competitive binding in the
presence of 10 μmol/L compound

| | | Final | |
|---|---|---|---|
| | Compound | concentration | 5-HT_{2A} [³H]-Ketanserin |
| Seq. | No. | umol/L | inhibition percentage % |
| 1 | II-1 | 10 | 99.5 |
| 2 | II-3 | 10 | 100 |
| 3 | II-4 | 10 | 99.9 |
| 4 | II-6 | 10 | 100 |
| 5 | II-8 | 10 | 100 |
| 6 | II-9 | 10 | 99.3 |
| 7 | II-11 | 10 | 99.6 |
| 8 | II-12 | 10 | 99.5 |
| 9 | II-14 | 10 | 99.7 |
| 10 | II-17 | 10 | 101 |
| 11 | III-2 | 10 | 99.1 |
| 12 | IV-3 | 10 | 92.8 |
| 13 | IV-4 | 10 | 97.4 |

2) IC₅₀ and Ki Values of Compounds with High Affinity

Seven compounds that exhibited high affinity in preliminary screening were subjected to concentration gradient test to determine their IC₅₀ and Ki values. Results were shown in Table 2.

TABLE 2
IC₅₀ and Ki value of the affinity between
the compound and 5-HT_{2A}receptor

| Compound | IC₅₀ result (nM) | Ki value (nM) |
|---|---|---|
| Aripiprazole | 3.7631 | 1.9806 |
| II-3 | 1.6590 | 1.1167 |
| II-6 | 4.7807 | 3.2178 |
| II-8 | 5.3290 | 2.7864 |
| II-9 | 2.9223 | 1.9670 |
| II-14 | 3.0920 | 2.0614 |
| II-17 | 3.6319 | 2.4445 |
| III-2 | 3.9937 | 2.6881 |

Seven compounds, namely compounds II-3, II-6, II-8, II-9, II-14, II-17 and III-2, have potent inhibitory activities on 5-HT_{2A} with a potency comparable to that of Aripiprazole.

### Example 26

### Inhibition on 5-HT Reuptake

The methodology of studying reuptake of monoamine neurotransmitters by brain synapses reported in Biochem Phearmacol Vol 22, pp 311-322 (1973) was used, which is currently one of the major means to perform studies on central neuropharmocology. The method can be used not only for the study of a drug's mechanism of action, but also for the screening of new drugs acting on such mechanisms. The invention utilized the methodology of studying reuptake of monoamine neurotransmitter 5-HT by brain synapses to investigate the inhibition of the compounds on 5-HT reuptake by brain synapses using Venlafaxine, the effective dual inhibitor of 5-HT and NA reuptake, as a positive control. The method was as follows:

### 1. Preparation of Rat Brain Synapses

Male SD rats were killed by cervical dislocation, heads were immediately cut and brains were obtained and put on ice, related brain tissues were dissected (forehead cortex was obtained for [³H]5-HT, [³H]NA reuptake test, corpus striatum was obtained for [³H]DA reuptake).

After the sample was weighed, 10 folds (V/W) ice cold 0.32 mol/L sucrose solution was added. The mixture was homogenized by teflon glass homogenizer; the resulting homogenate was centrifuged at 1000 g for 10 min at 4° C.; supernatant was obtained and centrifuged at 17000 g for 20 min at 4° C.; precipitate was collected and suspended in 30-fold volume of KRH Buffer (125 mM NaCl, 4.8 mM KCl, 1.2 mM CaCl₂, 1.2 mM MgSO₄, 1.0 mM KH₂PO₄, 22 mM HaHCO₃, 25 mM HEPES, 10 mM Glucose, 10 μM Pargyline, 0.2 mg/ml Ascorbic Acid), and put into ice bath until use.

### 2. [³H] 5-HT Reuptake Test

According to [a. Biochem Phearmacol Vol 22, pp 311-322 (1973); b. Marcel Dakker, Methods in Neurochemistry, I Vol. 2, New York, pp 1-52 (1972)], the stock solution of test sample was thawed prior to use and diluted with KRH Buffer to 100 μmol/L. A 50 μl aliquot was added to a 500 μl reaction system at a final concentration of 10 μmol/L. Then 50 μl suspended synapse membrane was added and mixed, incubated in water bath at 37° C. for 30 min; 10 nmol/L[3H] 5-HT was added and after incubation in water bath at 37° C. for 10 min, the reaction mixture was immediately taken out of the water bath and 2 ml ice cold 150 mmol/L Tris-HCl was added to terminate the reaction. The sample was collected on a round glass fiber filter by vacuum filtration and washed three times with 3 ml ice cold Tris-HCl buffer. The filter membrane was removed, dried in a far-infrared oven for 15 min, then transferred into a EP tube, in which 1.5 ml scintillation fluid was added, left overnight and detected by a scintillation counter. The test sample was not added into solvent controlled overall binding tube or non-specific binding tube. 50 μl solvent was added into the overall binding tube, and in [³H] 5-HT reuptake test, 600 μmol/L cocaine was added into the non-specific binding tube.

### 3. Test Results

The inhibition percentage results on 5-HT reuptake at identical concentration (0 1 mmol/L for both control drug and test drug) using Dutoxetine as a positive control were shown in Table 3.

TABLE 3
Inhibitory effects of the compounds on
serotonin (5-HT) reuptake

| | Final | 5-HTU [³H] 5-HT | |
|---|---|---|---|
| Compound | concentration | reuptake inhibition percentage % | |
| name | umol/L | Mean | SD |
| Dutoxetine | 10 | 90.57 | 1.61 |
| II-3 | 10 | 95.9 | 2.01 |
| II-6 | 10 | 96.71 | 1.73 |
| II-8 | 10 | 106.6 | 2.09 |
| II-9 | 10 | 105.3 | 2.97 |
| II-14 | 10 | 88.27 | 2.37 |
| III-2 | 10 | 86.01 | 1.83 |

Six compounds, namely compounds II-3, II-6, II-8, II-9, II-14, III-2, have potent inhibitory activities on 5-HT reuptake at 10 μmol/L with a potency comparable to that of Dutoxetine.

### Example 27

### Acetic Acid-Induced Abdominal Writhing Test in Mice

### 1. Experimental Animals

Kunming mice, SPF KM mice purchased from SHANGHAI SLAC LABORATORY ANIMAL CO. LTD and kept in normal environment.

### 2. Modes of Administration

The compounds were dissolved in water for injection at a concentration of 4 mg/ml, 2 mg/ml and 1 mg/ml and were given intragastrically to animals.

### 3. Doses

Three different doses (10, 20, and 40 mg/kg) were administered to the test groups.

### 4. Test Method

Aspirin was used as a positive control and acetic acid writhing test was used.

### 5. Experimental Protocol

30 mice (half male and half female) weighing 18-23 g were divided into five groups, including negative control group, positive control group, low dose group, medium dose group and high dose group:

| | | |
|---|---|---|
| negative control group | physiological saline | 20 ml/kg |
| positive control group | aspirin | 200 mg/kg |
| low dose group | test drug | 10 mg/kg |
| medium dose group | test drug | 20 mg/kg |
| high dose group | test drug | 40 mg/kg |

Mice in test group received test drug (10 mg/kg, 20 mg/kg, 40 mg/kg) via intragastric administration. Negative control group received physiological saline (20 ml/kg) via oral administration. Positive control group received aspirin (200 mg/kg) via oral administration. One hour later, each group received 0.7% acetic acid 10 ml/kg intraperitoneally. Five minutes later, the number of writhes was counted within the subsequent 15 min period; inhibition percentage of writhing response in each test group was calculated by the following equation:

$$Inhibition percentage = \frac{\begin{matrix}average number of writhes in negative control group - \\ average number of writhes in treatment group\end{matrix}}{average number of writhes in negative control group} \times 100 \%$$

6. Results of Multiple Dose Administration were Shown in Table 3.

TABLE 3
Screening results in acetic acid writhing test

| | Dose | | | | |
|---|---|---|---|---|---|
| | Inhibition percentage of | | | | |
| | writhing response (%) | | | | |
| Desig- | Aspirin | 10 | 20 | 40 | |
| nation | 200 mg/kg | mg/kg | mg/kg | mg/kg | Remarks |
| II-3 | 95.05** | 98.65** | 100.0** | 100.0** | intragastric |
| | | | | | administration |
| II-6 | 95.05** | 74.09* | 69.55 | 100.0** | intragastric |
| | | | | | administration |
| II-8 | 95.05** | 95.88* | 96.46** | 100.0** | intragastric |
| | | | | | administration |
| II-9 | 95.05** | 100** | 99.05* | 100** | intragastric |
| | | | | | administration |
| II-14 | 95.05** | 77.70* | 69.03 | 85.66** | intragastric |
| | | | | | administration |
| II-17 | 95.05** | 84.18* | 82.14* | 91.33** | intragastric |
| | | | | | administration |
| III-2 | 95.05** | 97.61* | 100.0** | 100.0** | intragastric |
| | | | | | administration |
| IV-4 | 95.05** | 88.33 | 99.44 | 97.78 | intragastric |
| | | | | | administration |

Note:
*p < 0.05,
**p < 0.01 VS negative control group

### Example 28

### Hot-Plate Test in Mice

### 1. Experimental Animals

Kunming mice, SPF KM mice were purchased from SHANGHAI SLAC LABORATORY ANIMAL CO. LTD and kept in normal environment.

### 2. Modes of Administration

The compounds were dissolved in water for injection at a concentration of 4 mg/ml, 2 mg/ml and 1 mg/ml and were given subcutaneously to animals.

### 3. Doses

Three different doses (10, 20, and 40 mg/kg) were administered to the test groups.

### 4. Test Method

Morphine was used as a positive control and hot plate test was used.

### 5. Experimental Protocol

30-40 mice (half male and half female) weighed from 18 to 23 gram were used. First, each mouse was placed on a hot plat at 55° C. to determine basic pain threshold for 2-3 times. Those animals with basic pain threshold of 5-30 s were qualified, unqualified mice were not used. 30 qualified mice were divided into five groups, including negative control group, positive control group, low-dose group, medium-dose group and high-dose group:

| negative control group | directly determine the basic pain threshold | | |
|---|---|---|---|
| positive control group | morphine | 0.2 mg/ml | 0.2 ml |
| low-dose group | test drug | 1 mg/ml | 0.2 ml |
| median-dose group | test drug | 2 mg/ml | 0.2 ml |
| high-dose group | test drug | 4 mg/ml | 0.2 ml |

Mice in test groups received test sample solution (10 mg/kg, 20 mg/kg, 40 mg/kg) via subcutaneous injection in the neck. Positive control group received subcutaneous injection of morphine (2 mg/kg). One hour later, pain threshold values were determined for mice in each group as post-drug pain threshold. Percentage increase in pain threshold was calculated according to the following equation:

$$Percentage increase in pain threshold \% = \frac{pain threshold after treatment - average basic pain threshold}{average basic pain threshold} \times 100 \% .$$

6. Results of Some Compounds were Shown in Table 5.

TABLE 5
Screening results in hot plate test in mice

| | Dose | | | | |
|---|---|---|---|---|---|
| | Percentage increase in pain threshold (%) | | | | |
| | Morphine | 10 | 20 | 40 | |
| Designation | 2 mg/kg | mg/kg | mg/kg | mg/kg | Remarks |
| II-3 | 202.5** | 95.48* | 194** | 133 | S.c. |
| II-6 | 202.5** | 41.11 | 73.68* | 72.38* | S.c. |
| II-8 | 202.5** | 97.49 | 160.50** | 172.81* | S.c. |
| II-9 | 202.5** | 97.98* | 220.49** | 229.39** | S.c. |
| III-2 | 202.5** | 92.55* | 172.2* | 97.22 | S.c. |
| IV-4 | 202.5** | 51.63 | 79.36* | 89.96* | S.c. |

Note:
*p < 0.05,
**p < 0.01 VS negative control group;
S.c.: subcutaneous

### Example 29

### Sedative Effects of the Compounds in Mice

Spontaneous activities of the mice were recorded by alternating current tube, Sedative effects were tested after single dose (20 mg/kg). Results were shown in Table 4.

TABLE 4
Screening results of compounds' sedative effects

| Designation | Sedative effect | Designation | Sedative effect |
|---|---|---|---|
| II-1 | 43* | II-12 | 52* |
| II-2 | 44* | II-13 | 66* |
| II-3 | 35 | II-14 | 20 |
| II-4 | 91** | III-1 | 54* |
| II-5 | 65* | III-2 | 64** |
| II-6 | 75** | III-3 | 52* |
| II-7 | 96** | IV-1 | 20 |
| II-8 | 42* | IV-2 | 52* |
| II-9 | 47* | IV-3 | 67* |
| II-10 | 99** | IV-4 | 70.8* |
| II-11 | 0 | | |

Note:
*P value < 0.05,
**P value < 0.01

### Example 30

### Competitive Binding Between the Compounds and Opioid Receptor Subtypes μ, δ, κ

Competitive binding between the compounds and opioid receptor subtypes μ, δ, κ was determined by radioactive ligand binding assay to verify that such compounds had non-opioid analgesic pathway.

Competitive binding assay was performed in overall binding tube, non-specific binding tube and sample tube respectively. 30 μg membrane protein, and [3H]Diprenorphine (final concentration of 0.4 nM) were added to the overall binding tube and the volume was adjusted to 200 μL with 50 mM Tris-HCl (pH7.4). 10 μM Naloxone was additionally added to the corresponding non-specific binding tube. The respective test compounds were added to the sample tube (final concentration of 10⁻⁵M), incubated at 37° C. for 30 min and placed in ice bath to terminate the reaction. The reaction mixture was filtered through GF/C (Whatman) glass fiber filter by vacuum filtration on a Millipore sample collector. The filter was washed three times with 50 mM Tris-HCl (pH7.4), each with 4 ml; dried and transferred into a 0.5 ml Eppendorf tube, in which 0.5 ml lipophilic scintillation fluid was added. Radioactivity was detected by a LS6500 scintillation counter. Each concentration had three duplicate test tubes and each separate test was repeated 3 to 4 times.

Specific binding CMP value for each sample tube=overall binding CPM value for each sample tube−CPM value of non-specific binding tube.

[Inhibition percentage of competitive binding between the test compound and different opioid receptor subtypes (%)=(100%−specific binding (CPM value)of sample tube/specific binding(CPM value)of solvent group×100%)]

Average was taken for each test drug from three duplicate tubes; each test was repeated two or more times. Data were presented as mean±SE and statistical comparison was made by ANOVA. None of the 5 tested compounds showed high affinity to the three different opioid receptor subtypes. Results were shown in Table 6.

TABLE 6
The affinity of compounds to opioid receptor subtypes μ, δ, κ

| | Tested | | | |
|---|---|---|---|---|
| | concentration | μ | δ | κ |
| Compound | (mol/L) | (%) | (%) | (%) |
| Naloxone | 10⁻⁶ | 100 | 100 | 100 |
| II-9 | 10⁻⁵ | 53.4 ± 0.7 | 0 | 28.5 ± 1.3 |
| II-14 | 10⁻⁵ | 41.9 ± 0.5 | 0 | 7.0 ± 0.6 |
| III-2 | 10⁻⁵ | 59.3 ± 0.7 | 0 | 44.1 ± 0.2 |
| IV-4 | 10⁻⁵ | 34.3 ± 1.3 | 0 | 37.3 ± 1.7 |

### Example 31

### Study on II-9 Acute Toxicity

Statistics were made according to Bliss method (“Experimental Design and Statistical Basis for Drug Evaluation”, Changxiao Liu, Ruiyuan Su, first edition, Military Medical Science Press, 1993, 80-90). The LD₅₀ after single intragastric administration of II-9 to mice is 800 mg/kg.

### Example 32

### Bacterial Reverse Mutation Test on II-9

Bacterial reverse mutation test on compound I-20 was performed on Salmonella typhimurium histidine auxotroph mutants TA97, TA98, TA100 and TA102 (purchased from MolTox company) using conventional procedures of Ames test.

Observation period: colony counting was done 48 hours after cultivation at 37° C.

Drug solutions with different concentrations were prepared with double distilled water, and the doses were 5, 50, 500, 1000, 5000 μg/plate.

Direct effect of the test drug in the absence of metabolic activity was determined by standard plate incorporation assay. The composition of the test top layer was: 2.0 ml top layer, 0.1 ml drug solution, 0.1 ml bacteria solution and 0.5 ml phosphate buffer.

Pre-incubation was used in the determination of the drug's mutagenic effect in the presence of metabolic activity. The composition of the test top layer is: 2.0 ml top layer, 0.1 ml drug solution, 0.1 ml bacteria solution and 0.5 ml S₉ mix.

The resulting drug solution, bacterial solution and S9 mix were first incubated at 35° C. for 30 min while shaking, and then tested according to standard plate incorporation assay. Each dose was tested in three plates, each mutant was tested in the absence or presence of metabolic activity (−S₉ or +S₉) and repeated twice, the number of revertant colonies was calculated as x±SD.

Results: the test included two parts, −S₉ and +S₉. TA98 in −S₉ test system and TA97 in +S₉ test system had bacteristatic effects. Other doses had no bacteristatic effect to all strains, and the background growth was good. None of the tested dose resulted in significant increase in the number of revertant colonies either in −S₉ system or in +S₉ system. Consequently, Ames test exhibited negative.

The results described above indicate that II-9 have obvious analgesic effect and can be well absorbed via oral administration. II-9 has no obvious affinity to opioid receptor subtypes μ, δ, κ, indicating its non-opioid analgesic pathway. With negative result in Ames test and high therapeutic index, II-9 has the potential to be developed as a novel non-opioid analgesic.

1. A benzisoxazolyl piperidine derivative having the following general formula, and a salt or a hydrate thereof: 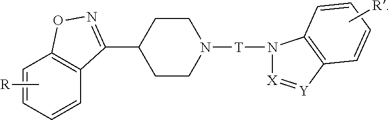
wherein:
R represents H, halogen, unsubstituted or halogen-substituted C₁-C₄ alkyl or unsubstituted or halogen substituted C₁-C₄ alkoxy;
X and Y represent independently CH or N;
R′ represents H, halogen, cyano, C₁-C₄ alkyl unsubstituted or substituted with halogen or cyano, C₁-C₄ alkoxy unsubstituted or substituted with halogen or cyano, or —C(═O)R₁, wherein R₁ represents H, C₁-C₄ alkyl or C₁-C₄ alkoxy; and
T represents a saturated or unsaturated, linear or branched carbon chain linking group having 1-10 carbon atoms, wherein any carbon atom of the carbon chain linking group can be replaced with one or more oxygen or sulfur atoms.

2. The benzisoxazolyl piperidine derivative according to claim 1, wherein R represents H, halogen, or C₁-C₄ alkoxy, preferably H, F or OCH₃.

3. The benzisoxazolyl piperidine derivative according to claim 1, wherein R′ represents H, halogen, cyano or —C(═O)R₁, wherein R₁ represents H, C₁-C₄ alkyl or C₁-C₄ alkoxy.

4. The benzisoxazolyl piperidine derivative according to claim 1, wherein R′ represents H, Cl, F, CN or COOCH₃.

5. The benzisoxazolyl piperidine derivative according to claim 1, wherein T represents a saturated, linear or branched carbon chain linking group having 2-7 carbon atoms, wherein any carbon atom of the carbon chain linking group can be replaced with one or more oxygen or sulfur atoms, preferably with one or two oxygen or sulfur atoms.

6. The benzisoxazolyl piperidine derivative according to claim 1, wherein the salt is hydrochloride, hydrobromide, sulfate, trifluoroacetate or methanesulfonate, preferably hydrochlorid or hydrobromide.

7. The benzisoxazolyl piperidine derivative according to claim 1, wherein the salt contains 0.5-3 molecules of crystal water.

8. The benzisoxazolyl piperidine derivative according to claim 1, being a compound selected from the group consisting of
3-(1-(2-(1H-indol-1-yl)ethyl)piperidin-4-yl)-6-fluorobenzo[d]isoxazole,
3-(1-(3-(1H-indol-1-yl)propyl)piperidin-4-yl)-6-fluorobenzo[d]isoxazole,
3-(1-(4-(1H-indol-1-yl)butyl)piperidin-4-yl)-6-fluorobenzo[d]isoxazole,
3-(1-(5-(1H-indol-1-yl)pentyl)piperidin-4-yl)-6-fluorobenzo[d]isoxazole,
6-fluoro-3-(1-(3-(6-fluoro-1H-indol-1-yl)propyl)piperidin-4-yl)benzo[d]isoxazole,
1-(3-(4-(6-fluorobenzo[d]isoxazol-3-yl)piperidin-1-yl)propyl)-1H-indole-6-carbonitrile,
methyl 1-(3-(4-(6-fluorobenzo[d]isoxazol-3-yl)piperidin-1-yl)propyl)-1H-indole-6-carboxylate,
6-fluoro-3-(1-(4-(6-fluoro-1H-indol-1-yl)butyl)piperidin-4-yl)benzo[d]isoxazole,
1-(4-(4-(6-fluorobenzo[d]isoxazol-3-yl)piperidin-1-yl)butyl)-1H-indole-6-carbonitrile,
3-(1-(4-(6-chloro-1H-indol-1-yl)butyl)piperidin-4-yl)-6-fluorobenzo[d]isoxazole,
3-(1-(3-(1H-benzo[d]imidazol-1-yl)propyl)piperidin-4-yl)-6-fluorobenzo[d]isoxazole,
3-(1-(4-(1H-benzo[d]imidazol-1-yl)butyl)piperidin-4-yl)-6-fluorobenzo[d]isoxazole,
3-(1-(3-(1H-indazol-1-yl)propyl)piperidin-4-yl)-6-fluorobenzo[d]isoxazole,
1-(4-(4-(6-fluorobenzo[d]isoxazol-3-yl)piperidin-1-yl)butyl)-1H-indazole-6-carbonitrile,
1-(4-(4-(benzo[d]isoxazol-3-yl)piperidin-1-yl)butyl)-1H-indole-6-carbonitrile,
6-fluoro-3-(1-(3-(6-fluoro-1H-benzo[d]imidazol-1-yl)propyl)piperidin-4-yl)benzo[d]isoxazole,
6-fluoro-3-(1-(4-(6-fluoro-1H-benzo[d]imidazol-1-yl)butyl)piperidin-4-yl)benzo[d]isoxazole,
1-(4-(4-(6-fluorobenzo[d]isoxazol-3-yl)piperidin-1-yl)butyl)-1H-benzo[d]imidazole-6-carbonitrile,
1-(2-(4-(6-fluorobenzo[d]isoxazol-3-yl)piperidin-1-yl)ethoxy)-1H-indole-6-carbonitrile,
1-(3-(4-(6-fluorobenzo[d]isoxazol-3-yl)piperidin-1-yl)propoxy)-1H-indole-6-carbonitrile,
3-(1-(2-(6-chloro-1H-benzo[d][1,2,3]triazol-1-yloxy)ethyl)piperidin-4-yl)-6-fluorobenzo[d]isoxazole, or
3-(1-(3-(6-chloro-1H-benzo[d][1,2,3]triazol-1-yloxy)propyl)piperidin-4-yl)-6-fluorobenzo[d]isoxazole,
or a salt or a hydrate thereof.

9. The benzisoxazolyl piperidine derivative according to claim 8, the derivative being a compound selected from the group consisting of
6-fluoro-3-(1-(4-(6-fluoro-1H-indol-1-yl)butyl)piperidin-4-yl)benzo[d]isoxazole,
1-(4-(4-(6-fluorobenzo[d]isoxazol-3-yl)piperidin-1-yl)butyl)-1H-indole-6-carbonitrile, or
6-fluoro-3-(1-(4-(6-fluoro-1H-benzo[d]imidazol-1-yl)butyl)piperidin-4-yl)benzo[d]isoxazole,
or a salt or a hydrate thereof.

10. A pharmaceutical composition comprising a therapeutically effective amount of a benzisoxazolyl piperidine derivative according to claim 1, a salt or a hydrate thereof, and a pharmaceutically acceptable carrier.

11. Use of a benzisoxazolyl piperidine derivative according to claim 1 in the preparation of analgesics and sedatives for the treatment of pains.

12. The use according to claim 11, wherein the pains include nociceptive pain, acute pain, chronic pain, neuropathic pain, psychogenic pain and mixed pain.

13. A method of treating pains in mammals, comprising administering a benzisoxazolyl piperidine derivative according to claim 1, a salt or a hydrate thereof to an individual with such need.

14. The method according to claim 13, wherein the pains include nociceptive pain, acute pain, chronic pain, neuropathic pain, psychogenic pain and mixed pain.
